(19) United States
(12) Patent Application Publication
(10) Pub. No.: US 2010/0204622 A1
(43) Pub. Date: Aug. 12, 2010

(54) ANALYSIS METHOD OF A BODY TYPE, AND CARDIOPULMONARY RESUSCITATION APPARATUS USING THE SAME

(76) Inventors: Sungoh Hwang, Gangwon-do (KR); Wonchul Kim, Gyeonggi-do (KR)

Correspondence Address: DICKSTEIN SHAPIRO LLP, 1825 EYE STREET NW, Washington, DC, 20006-5403, US

(21) Appl. No.: 12/677,671

(22) PCT Filed: Sep. 5, 2008
(86) PCT No.: PCT/KR08/05261

§ 371 Date: Mar. 11, 2010

(30) Foreign Application Priority Data
Sep. 11, 2007 (KR) 10-2007-0092284

(51) Int. Cl. A61H 31/00; A61B 5/103

(52) U.S. Cl. 601/41; 600/587

(57) ABSTRACT

Disclosed is an analysis method of a body type and a cardiopulmonary resuscitation apparatus using the same, the cardiopulmonary resuscitation apparatus includes: a girth measuring unit that measures the victim's girth on the basis of length change of the thorax band by the length adjusting unit; a body type analyzing unit that selects an inverse formula corresponding the victim's characteristic information provided from the outside and analyzing the victim's body type by substituting the girth to the inverse formula; a control calculating unit that calculates appropriate compression depth and artificial tidal volume of the victim by substituting the body type information obtained by the body type analyzing unit to a corresponding conversion formula; and a MICOM that controls the compressing unit and the respiring unit on the basis of the compression depth and tidal volume.

### TECHNICAL FIELD

The present invention relates to an analysis method of a body type and a cardiopulmonary resuscitation apparatus using the same, particularly a method of analyzing body type information of a victim of cardiac arrest and a cardiopulmonary resuscitation apparatus that can accurately adjust chest compression depth and tidal volume according to the victim's body type, using the method.

### BACKGROUND ART

In general, cardiopulmonary resuscitation, which is an emergency medical procedure supplying oxygenated bloodstream to the entire body of a victim of heart arrest, instead of the heart and lung, is divided into artificial blood circulation that makes bloodstream by compressing the chest and artificial respiration for helping respiration.

The coronary perfusion pressure should be maintained at least at 20 mmHg or more to generate spontaneous bloodstream circulation in cardiopulmonary resuscitation.

However, a method of repeatedly compressing only the breastbone in the related art could only generate about 17 to 23% of normal heart blood flow and was insufficient in restoring spontaneous circulation. Accordingly, a variety of cardiopulmonary resuscitation apparatuses have been proposed to increase the blood flow.

A patent relating to a cardiopulmonary resuscitation apparatus that can supply larger blood flow to the brain and heart by constricting the thorax while compressing the chest, was applied by the applicant(s) on Jul. 2, 1998, and was registered on Aug. 3, 2000 in Korea (Patent Registration No. 10-0270596).

Further, patents relating to cardiopulmonary resuscitation apparatuses for improving the cardiopulmonary resuscitation apparatus disclosed in Patent Registration No. 10-0270596, that is, cardiopulmonary resuscitation apparatuses of which the lengths of thorax bands can be easily adjusted according to the physique of a victim, were applied by the applicant(s) on Sep. 21, 2001 and Mar. 28, 2002, respectively, and were registered in Korea (Patent Registration Nos. 10-0413009, 10-0448449).

Further, a patent relating to an apparatus that is useful in transporting a patient and making it possible to perform cardiopulmonary resuscitation within a short time and continuously while transporting the patient, was applied by the applicant(s) on Feb. 20, 2003, and was registered in Korea (Patent Registration No. 10-0517298).

Further, patents relating to a circuit for controlling operation of a cardiopulmonary resuscitation apparatus that can easily control thorax compression and respiration, by using air pressure in the cardiopulmonary resuscitation apparatus, and a cardiopulmonary resuscitation apparatus that includes a controller for simply and accurately controlling the amount of chest compression, by using sound in the cardiopulmonary resuscitation apparatus, were applied by the applicant(s) on Apr. 28, 2005 and Apr. 25, 2006, respectively, and were registered in Korea (Patent Registration Nos. 10-0633799, 10-0706701).

Meanwhile, according to the apparatuses disclosed in Patent Registration Nos. 10-0633799 and 10-0706701, a user adjusts chest compression depth and tidal volume that are applied to a victim at his/her discretion, and the cardiopulmonary resuscitation apparatuses have been designed to be operated at the discretion of the user of the chest compression depth and tidal volume without considering the physical characteristics of a victim.

Therefore, according to the cardiopulmonary resuscitation apparatuses that are operated at the discretion of the user in the related art, it was difficult to take an appropriate measure in an emergency because the chest compression depth and tidal volume are different according to physical characteristics of a victim, such as sex and age.

Further, because it is difficult to find out the physical characteristics from the victim, it is required to automatically estimate physical characteristics and perform appropriate cardiopulmonary resuscitation.

### DISCLOSURE OF INVENTION

### Technical Problem

In order to overcome the problems in the related art, an object of the invention is to provide an analysis method of a body type that analyzes information about the body type on the basis of characteristic information and body size of a victim.

Further, another object of the invention is to provide a cardiopulmonary resuscitation apparatus that can perform accurate cardiopulmonary resuscitation by automatically adjusting optimal chest compression and tidal volume according to the physical characteristics.

### Technical Solution

In order to achieve the objects of the invention, an analysis method of body type according to a preferred embodiment of the invention includes receiving information of characteristic information and body sizes of a person whose body type information is analyzed; classifying the body type of the person according to the characteristic information; selecting an inverse formula corresponding to the classified body type; calculating body type information of the person by substituting the received body sized to the selected inverse formula; and analyzing the body type of the person on the basis of the calculated body type information.

A cardiopulmonary resuscitation apparatus, which includes a thorax band that tightens and contracts the victim's thorax, and a length adjusting unit that adjusts length of the thorax band using normal/reverse rotation of both side bobbins that are engaged with gears connected with the thorax band, according to the invention, includes a girth measuring unit that measures the victim's girth on the basis of length change of the thorax band by the length adjusting unit, in which the girth measuring unit includes: a variable gear assembly that is engaged with a gear at a side of the length adjusting unit; a length change measuring part that measures change in length of the thorax band by detecting normal/reverse rotation of the variable gear assembly; and a girth calculating part that calculates the victim's girth on the basis of the length change of the thorax band measured by the length change measuring part.

Further, a cardiopulmonary resuscitation apparatus, which includes a thorax band that tightens and contracts the victim's thorax when a chest compressing unit compresses the chest, a length adjusting unit that adjusts length of the thorax band using normal/reverse rotation of both side bobbins that are engaged with gears connected with the thorax band, and a respiring unit for artificial respiration, according to a preferred embodiment of the invention, includes: a girth measuring unit that measures the victim's girth on the basis of length change of the thorax band by the length adjusting unit; a body type analyzing unit that selects an inverse formula corresponding the victim's characteristic information provided from the outside and analyzing the victim's body type by substituting girth to the inverse formula; a control calculating unit that calculates appropriate compression depth and artificial tidal volume of the victim by substituting the body type information obtained by the body type analyzing unit to a corresponding conversion formula; and a MICOM that controls the compressing unit and the respiring unit on the basis of the compression depth and tidal volume.

A method of cardiopulmonary resuscitation using the cardiopulmonary resuscitation apparatus according to an embodiment of the invention includes: measuring the victim's girth on the basis of length change of the thorax band by the length adjusting unit; receiving characteristic information about the victim; analyzing body type of the victim by selecting a corresponding inverse formula on the basis of the characteristic information and analyzing the body type of the victim by substituting the girth to the inverse formula; control-calculating appropriate compression depth and artificial tidal volume for the victim by substituting the body type information obtained by the body type analyzing unit to a corresponding conversion formula; and performing cardiopulmonary resuscitation to the victim by controlling the compressing unit and the respiring unit on the basis of the compression depth and tidal volume.

### Advantageous Effects

Because the invention provides an analysis method of body type that can obtain the victim's body type information from the victim's characteristic information and body sizes, it is possible to grasp the victim's body type information without checking all of body sizes of the victim.

Further, according to the invention, it is possible to more accurately and quickly perform cardiopulmonary resuscitation because it is possible to optimize and automatically adjust chest compression and tidal volume according to the victim's body type information.

Further, according to the invention, since optimized sensors that detect the operational condition of the cardiopulmonary resuscitation apparatus are provided, it is possible to considerably improve efficiency in chest compression and artificial respiration.

On the other hand, the invention is not limited to the above embodiments and can be variously modified without departing from the scope of the invention, and it should be understood that the modified scope is included in the following claims.

### BRIEF DESCRIPTION OF THE DRAWINGS

FIG. 1 is a view illustrating the configuration of a cardiopulmonary resuscitation apparatus according to an embodiment of the invention.

FIG. 2 is a view illustrating exemplary operation of a cardiopulmonary resuscitation apparatus according to an embodiment of the invention.

FIG. 3 is a block diagram illustrating the configuration of a cardiopulmonary resuscitation apparatus according to a preferred embodiment of the invention.

FIG. 4 is a perspective view of a length adjusting unit, a thorax band, and a girth measuring unit according to an embodiment of the invention.

FIG. 5 is a block diagram simply illustrating a length adjusting unit, a thorax band, and a girth measuring unit according to an embodiment of the invention.

FIG. 6 is a view showing a compressing unit according to an embodiment of the invention.

FIG. 7 is a block diagram simply illustrating a compressing unit according to an embodiment of the invention.

FIG. 8 is an exploded view of a respiring unit according to an embodiment of the invention.

FIG. 9 is a block diagram simply illustrating a compression driving unit according to an embodiment of the invention.

FIG. 10 is a flowchart illustrating cardiopulmonary resuscitation according to an embodiment of the invention.

FIG. 11 is a flowchart illustrating a method of analyzing a body type according to a preferred embodiment of the invention.

### BEST MODE FOR CARRYING OUT THE INVENTION

A cardiopulmonary resuscitation apparatus according to a preferred embodiment of the invention is described hereafter with reference to the accompanying drawings

A cardiopulmonary resuscitation apparatus according to an embodiment of the invention, as disclosed in Patent Registration Nos. 10-0517298, 10-0633799, and 10-0706701, is detachably equipped in an ambulance or individual equipment and can automatically control chest compression and tidal volume that are applied to a victim, using a controller.

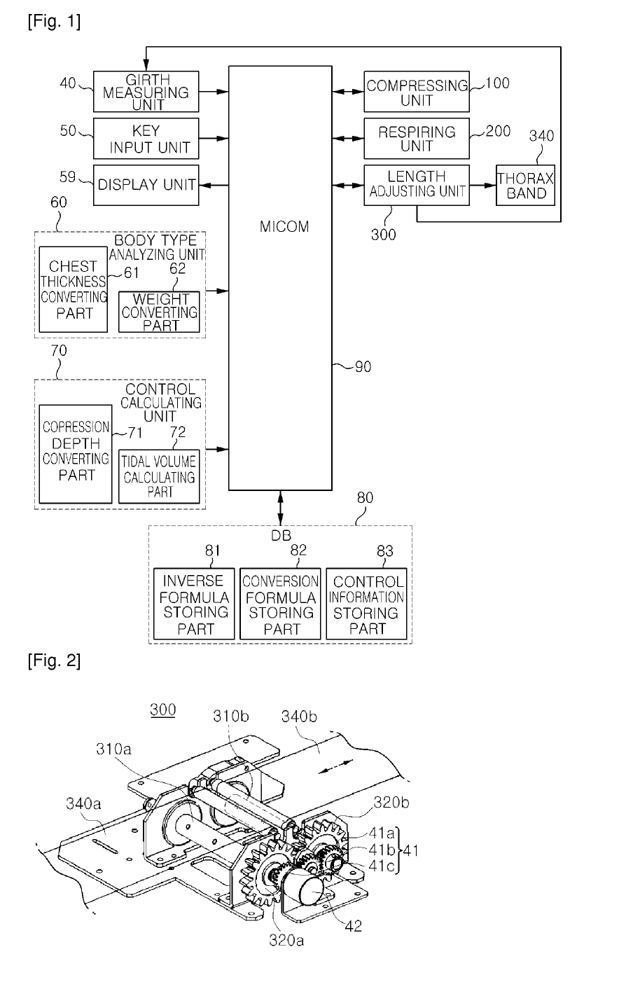

FIG. 1 is a block diagram illustrating the configuration of a cardiopulmonary resuscitation apparatus according to a preferred embodiment of the invention.

As shown in FIG. 1, the cardiopulmonary resuscitation apparatus according to a preferred embodiment of the invention includes: length adjusting unit 300 that adjust the length of a thorax band 340; a girth measuring unit 40 that measures the girth when the length of the thorax band 340 changes by the length adjusting unit 300; a key input unit 50 that receives characteristic information and various operation orders; a display unit 59 that displays operating conditions etc.; a body type analyzing unit 60 that analyzes information on the body type of a victim; and a control calculating unit 70 that calculates control information for cardiopulmonary resuscitation on the basis of the information on the body type; a DB 80; a MICOM 90; a compressing unit 100 that repeatedly compresses the victim's chest a predetermined number of times; and a respiring unit 200 that applies artificial respiration to the victim.

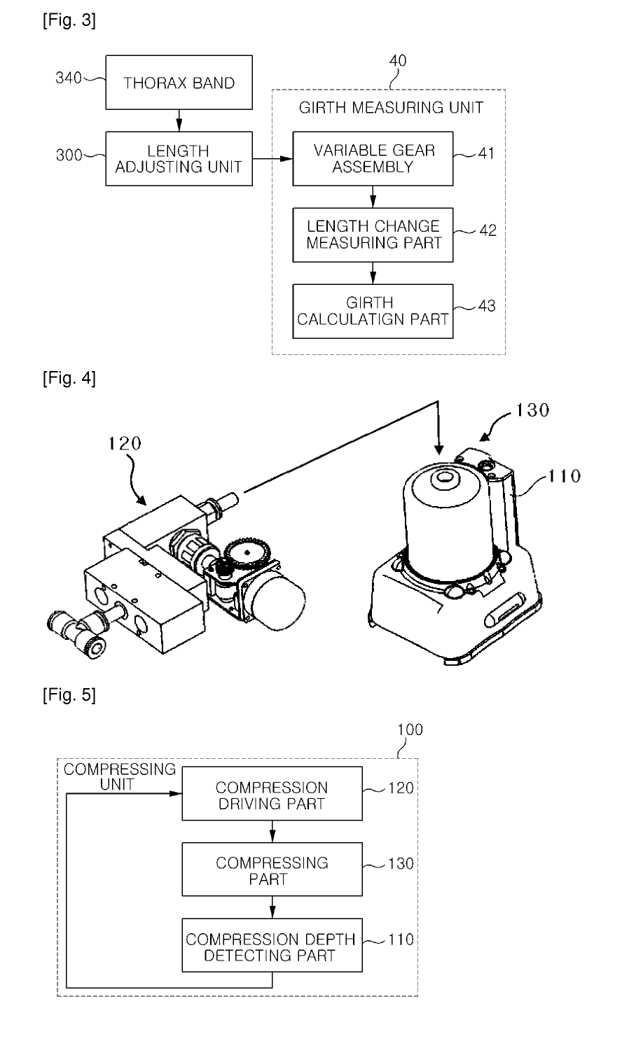

FIG. 2 is a perspective view of the length adjusting unit, thorax band, and girth measuring unit and FIG. 3 is a block diagram simply illustrating the length adjusting unit, thorax band, and girth measuring unit.

The length adjusting unit 300 adjusts the length of the thorax band 340 that is wound around the victim's thorax. For example, the length of the thorax band 340 can be adjusted to be the same as the victim's girth because the compressing unit 100 cannot sufficiently compress the victim's thorax when the thorax band 340 wound around the victim's chest is loose or tight.

That is, as shown in FIG. 2, the length adjusting unit 300 includes a pair of bobbins 310a, 310b that are engaged with gears 320a, 320b such that the left and right thorax bands 340a, 340b are always wound and loosened at the same length while being drawn in/out and a driving unit (not shown) that drives the pair of bobbins 310a, 310b. The driving unit, as disclosed in Patent Registration No. 10-0517298, can be configured to be able to drive the pair of bobbins 310a, 310b in normal and reverse direction.

The thorax band 340 is connected to the length adjusting unit 300 and detachable while being wound around the victim's chest, such that it can compress the chest when the victim's thorax is compressed by the compressing unit 100.

As shown in FIGS. 2 and 3, the girth measuring unit 40 measures the victim's girth on the basis of change in length of the thorax bands 340a, 340b by the length adjusting unit 300. The girth measuring unit 40 includes a variable gear assembly 41 that is engaged with a gear of the length adjusting unit 300, a length change adjusting part 42 that measures change in length of the thorax bands 340a, 340b by detecting change of the variable gear assembly 41, and a girth calculating part 43 that calculates the victim's girth on the basis of the change in length of the thorax bands 340a, 340b measured by the length change measuring part 42.

The variable gear assembly 41 is composed of a first gear 41a that is fitted on the end of a gear 320b of a bobbin 410b of the length adjusting unit 300, a second gear 41b that is engaged with the first gear 41a, and a third gear 41c that is engaged with the second gear and transmits the amount of normal/reverse rotation to the length change measuring part. That is, when the length adjusting unit 300 draws in/out the left and right thorax bands 340a, 340b, the pair of bobbins 310a, 310b are driven by driving force of the driving unit (not shown). As the gears 320a, 320b that are engaged with the pair of bobbins 310a, 310b are rotated in normal/reverse direction by the bobbins, the first, second, and third gears of the variable gear assembly 41 are rotated.

The length change measuring part 42 is a detecting sensor, such as a potentiometer. The length change measuring part 42 is connected with the third gear 41c of the variable gear assembly 41 to receive the amount of normal/reverse rotation with respect to the bobbin 310b of the length adjusting unit 300 when the length adjusting unit 300 draws in the thorax bands 340a, 340b. That is, the length change measuring part 42 outputs predetermined amount of detection (DC Resistance) according to the detected amount, and the outputted measured values are the changes in length of the thorax bands 340a, 340b.

Further, the length change measuring part 42 calculates the number of normal/reverse rotation of the bobbin 310b to allow several detecting means, which can measure changes in drawing-in length of the thorax bands 340a, 340b, such as a rotary encoder (not shown), to be used.

The girth calculating part 43 calculates the victim's girth from an equation of length change on the basis of the drawn-in length change of the thorax bands 340a, 343b measured by the length change measuring part 42. That is, the girth calculating part 42 calculates and outputs the victim's girth corresponding to the length change (DC Resistance) of the thorax band 340.

Equation of length change,

Y=aX2+bX+c

where a, b, and c are constants, X is resistance value, Y is girth, and the range is 67<=Y<=157 and 1500<=X<=4300, and the unit of the girth is cm).

The graph constructed in the following Table 1 shows the girth calculated from the equation of length change.

TABLE 1

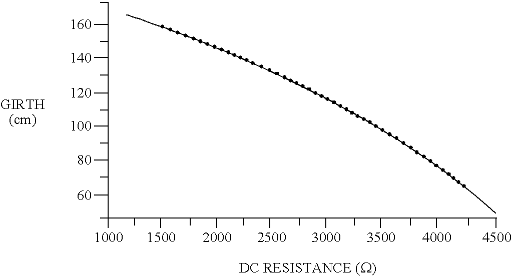

The graph constructed in Table 1 shows the ratio of length change of the thorax band (DC Resistance) and the girth calculated by the girth measuring part 40.

Further, the girth measuring part 40 of the invention can use other detecting sensors that can measure the length change of the thorax bands 340a, 340b, than the potentiometer exemplified above.

As described above, because the invention can automatically measure the victim's girth without separately checking it, it is possible to save time and ensure accuracy of the victim's girth.

The key input unit 50 receives orders needed for the operation of the cardiopulmonary resuscitation apparatus according to the invention or the victim's characteristic information. For example, the key input unit receives needed values from the victim's characteristic information (e.g. sex, age, flatness of chest) or various operation orders, such as turning on/off power the cardiopulmonary resuscitation apparatus 1.

Further, the key input unit 50 has a button (not shown) for an order that tightens or loosens the thorax band 340, a button (button) for mode selection, which selects a desired operation mode that can be operated in various ways, and a button (not shown) for selecting chest compression depths (in detail, thorax compression depths) in stages according to victims. The button for tightening or loosening the thorax band 340 is turned on by pressing once, such as a tact switch (tact s/w), which is selected to fasten from the On-state by pressing again, and then turned off and the fastening is off by pressing again from the fastening-state. Further, the mode selection button and the button for selecting chest compression depth may be replaced by a rotary switch, respectively.

Various operation modes may include a cardiopulmonary resuscitation mode that performs chest compression and artificial respiration, which is the basic cardiopulmonary resuscitation for a victim, at a predetermined ratio (e.g. chest compression (thirty times): artificial respiration (two times)), a continuous chest compression mode that continuously performs only chest compression for the victim, and a respiration mode that performs only artificial respiration while stopping the chest compression in the operation of continuously performing chest compression and artificial respiration for the victim.

Further, the key input unit 50 includes a remote controller for user's convenience. Therefore, the buttons are provided in the remote controller.

The body type analyzing unit 60 classifies body types on the basis of characteristic information of victims (e.g. sex and age), which is inputted by the key input unit 50. The body type analyzing unit 60 selects pre-calculated inverse formula from statistics about body types of general people, according to the classified body types. The body type analyzing unit 60 calculates other body type information (body size) of victims by substituting the victim's girth calculated by the girth measuring unit 40 to a selected inverse formula.

That is, the body type analyzing unit 60 calculates the victim's girth, weight, and height etc. by applying the inverse formula predetermined according to the victim's sex and age to the victim's girth.

The body type analyzing unit 60 is composed of a chest thickness converting part 61 that calculates the chest thickness on the basis of the victim's girth and a weight converting part 62 that calculates the weight on the basis of the victim's girth.

TABLE 2
Classification of Body Type

Characteristic Information

| Body Type | Sex | Age |
|---|---|---|
| A | Male | Teenage |
| A′ | Female | Teenage |
| B | Male | Twenties~Thirties |
| B′ | Female | Twenties~Thirties |
| C | Male | Forties~Fifties |
| C′ | Female | Forties~Fifties |
| D | Male | Fifties~Seventies |
| D′ | Female | Fifties~Seventies |

Table 2 shows body types with body characteristics classified according to the victim's characteristic information (sex, age, etc.). The exemplary body classification can be shown different according to the characteristic information.

Inverse Formula is,

chest thickness=k+l*girth, and

weight=m+n*girth

(where, k, l, m, and n become different according to constant body types (A, A′, B, B′, C, C′, D, D′) (e.g. Table 2) calculated according to sex and age). The chest thickness is in mm, the weight is in kg, and other body type information is in SI unit.

The inverse formulae are obtained from functional relationship between the girth and each index, in which the indexes are C-9 (chest thickness), F-10 (girth), A-6 (height), and H-4 (weight) in ‘National Anthropometric Survey of Korea’ published by Korean Agency for Technology and Standards. The inverse formula makes it possible to estimate body index needed for cardiopulmonary resuscitation from only some body information, such as girth.

The control calculating unit 70 calculates reference values (e.g. compression depth, tidal volume, respiration pressure etc.) needed for the operation of the cardiopulmonary resuscitation apparatus of the invention by substituting various body type information of a victim calculated by the body type analyzing unit 60 to a conversion formula pre-calculated from statistics in respect of the relationships between body shapes of general people and cardiopulmonary resuscitation. The control calculating unit 70 is composed of a compression depth converting part 71 that calculates compression depth on the basis of chest thickness and a tidal volume calculating part 72 that calculates tidal volume on the basis of the weight.

Conversion Formula:

compression depth=chest thickness*0.25, and

tidal volume=weight*6.5

(where the compression depth and tidal volume are in SI unit).

The conversion formula can convert the chest thickness and weight into the compression depth (chest thickness*0.25) and the artificial tidal volume (weight*6.5), on the basis of average of Descriptive Statistics.

On the average, the compression depth for women is 5 mm smaller than men and the artificial tidal volume for women is 65 ml smaller than men, such that it can be estimated that the difference between men and women is definitive.

Further, as for ages, it can be estimated that teenage and post-twenties are definitively differentiated between both men and women and the compression depth (girth) is not largely changed after the twenties, but the artificial tidal volume (weight) slightly increases after the twenties and then decreases.

Difference in compression depth between victims having a flat chest and a thick chest is about 5 mm for both men and women, and difference in the artificial tidal volume is about 9.75 ml for men and 8.125 ml for women. Therefore, it can be estimated that the difference between victims having flat and thick chests is not large.

TABLE 3
Compression Depth for Men/Women and Age

| | Men | | Women | |
|---|---|---|---|---|
| | Compression | | Compression | |
| Classification | Depth | Flat-Thick | Depth | Flat-Thick |
| Teenage | 46.5 | — | 42.75 | — |
| Twenties~ | 51.75~43.25 | 5.25~4.50 | 45.25~47.25 | 4.25~5.00 |
| Thirties | | | | |
| Forties~ | 54.00~55.25 | 4.50 | 49.25~51.75 | 4.25~5.00 |
| Fifties | | | | |
| Sixties~ | 55.25~55.00 | 4.50~5.00 | 52.75~53.50 | 5.50~7.00 |
| Seventies | | | | |

TABLE 4
Artificial Tidal Volume for Men/Women and Age

| | Men | | Women | |
|---|---|---|---|---|
| | Artificial | | Artificial | |
| Classification | Tidal Volume | Flat-Thick | Tidal Volume | Flat-Thick |
| Teenage | 370.5 | — | 318.5 | — |
| Twenties~ | 448.5~468.0 | 19.5~13.0 | 351.0~364.0 | 0~19.5 |
| Thirties | | | | |
| Forties~ | 455.0~448.5 | 0~6.5 | 370.5~390.0 | 6.5 |
| Fifties | | | | |
| Sixties~ | 422.5~396.5 | 6.5 | 377.0~357.5 | 13 |
| Seventies | | | | |

Table 3 and Table 4 exemplify compression depth and artificial tidal volume for men and women/ages obtained by the conversion formula on the invention.

Further, the inverse formula and conversion formula of the invention can be corrected depending on the victim's chest flatness, if needed.

As described above, the invention can apply appropriate amount of compression depth and artificial tidal volume for cardiopulmonary resuscitation according to the characteristic information (sex and age) of victims, such that accurate and effective cardiopulmonary resuscitation can be applied.

The DB 80 is a data base storing a variety of information needed for controlling the MICOM 90. The DB 80 can store classified data to remove, renew, and input a variety of stored data. The DB 80 is composed of an inverse formula storing part 81 that stores calculation formulae, a conversion formula storing part 82 that stores conversion formulae, and a control information storing part 83 that stores data needed for controlling the MICOM.

The MICOM 90 is micro controller unit (MCU) that controls various operations of the cardiopulmonary resuscitation apparatus 1. The MICOM 90 controls the operations of the compressing unit 100, the respiring unit 200, and the length adjusting unit 300, according to various body types of victims calculated by the control calculating unit 70.

Further, the MICOM 90 functions as a controller that controls chest compression and tidal volume of cardiopulmonary resuscitation apparatuses in the related art. That is, the MICOM 90, in addition to controlling the display operation of the display unit 59, makes a corresponding operation according to a mode selection signal inputted by pressing keys of the key input unit 50.

FIG. 4 is a view illustrating the compressing unit according to an embodiment of the invention and FIG. 5 is a block diagram simply illustrating the compressing unit according to an embodiment of the invention.

As shown in FIGS. 4 and 5, the compressing unit 100 compresses the victim's chest at a predetermined period using air pressure. For example, when the victim's heart stops, that is, the operation of the heart that circulates the blood to the brain and other parts is stopped, the compressing unit 100 compresses the thorax corresponding to the heart of the victim to make blood circulation.

The compressing unit 100 is composed of a compressing part 130 that compresses the victim's thorax, a compression driving part 120 that drives the compressing part 130, and a compression depth detecting part 110 that is equipped in the compression driving unit 120 and measures the compression depth of the victim's thorax.

The compressing part 130 is disposed on the victim's chest and compresses an objective part using driving force of the compression driving part 120.

The compression driving part 120 drives the compressing part 130 to compress an objective part by adjusting the driving force (e.g. gas pressure, such as oxygen and air, pumping pressure of a motor) which is supplied to the compressing part 130.

If it is possible to control pressurized-oxygen in response to a control signal transmitted from the MICOM 90, the compression driving part 120 can be composed of a compressing piston (not shown) that generates compression driving force, a directional control valve (not shown) that controls the compression and return of the compressing piston, and a FCV (Flow Control Valve) (not shown) that is an automatic control valve increasing/decreasing oxygen flow rate applied to the compressing piston. The compression may be adjusted by an opening/closing knob of the FCV using a stepper motor.

The compression depth detecting part 110, which is a senor disposed in the compressing part 130 and detects the degree of compression and return of the compressing part 130, measures compression depth of the victim's chest by detecting degree of compression and return of the compressing part 130.

The compression depth detecting part 110 can detect compression depth of the chest using a non-contacting sensor. The compression depth detecting part 110 can detect cycle distance of the compression piston (not shown) in a compression cylinder when compressing the victim's chest by embedding a permanent magnet in the compressing piston generating driving force of the compressing part 130 and disposing a hole sensor (not shown) and a display LED at a predetermined distance (e.g. 01 to 1 cm) on the outer surface of a compressing cylinder tube where the compressing piston is disposed, which makes it possible to detect compression depth of the chest.

The non-contacting sensor of the compression depth detecting part 110 shows stable detecting performance at a piston velocity of 0.166 m/sec for compression frequency of hundreds times per minute, while the compressing piston of the compressing part 100 may be manufactured in a anti-rotary type.

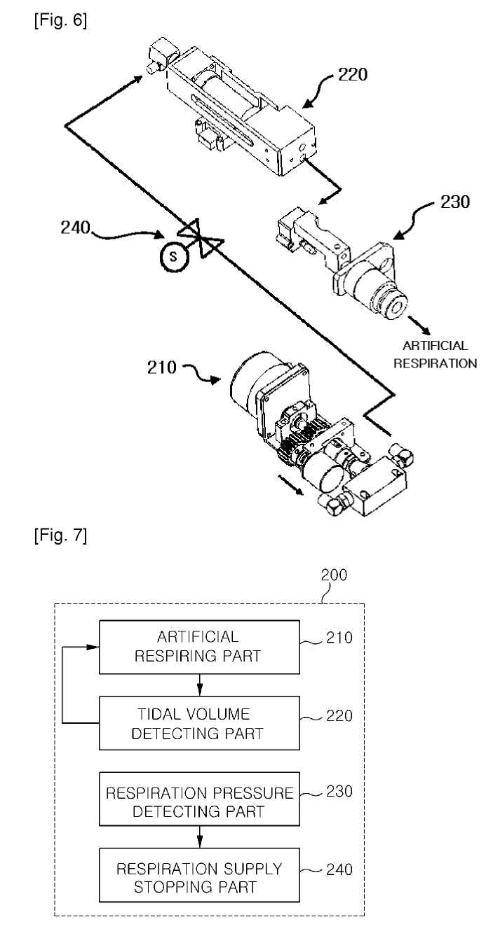

FIG. 6 is an exploded view showing the respiring unit according to an embodiment of the invention and FIG. 7 is a block diagram simply illustrating the compression driving part according to an embodiment of the invention.

As shown in FIGS. 6 and 7, the respiring unit 200 provides artificial respiration (e.g.

oxygen or air containing oxygen) to a victim. For example, even if a victim stops respiring, that is, spontaneous respiration is stopped, while blood circulation and heat beat continue for a short time, the respiring unit 200 performs artificial respiration for restarting spontaneous respiration by artificially supplying respiration oxygen through the victim's respiration hole (nose, mouth).

The respiring unit 200 is composed of an artificial respiring part 210 that discharges respiration oxygen, a tidal volume detecting part 220 that is disposed in the artificial respiring part 210 and measures the amount of discharged respiration oxygen, a respiration pressure detecting part 230 that measures the pressure of the respiration oxygen discharged through the artificial respiring part 210, and a respiration supply stopping part 240 that stops respiration oxygen supply before the pressure of the respiration oxygen measured by the respiration pressure detecting part 230 exceeds a predetermined limit.

The artificial respiring part 210 supplies respiration oxygen to a victim by adjusting the tidal volume at appropriate pressure. That is, the artificial respiring part 210 supplies respiration oxygen to the victim until the tidal volume reaches the level calculated in response to a control signal of the MICOM 90.

The tidal volume detecting part 220 is a sensor that detects the supply amount of respiration oxygen supplied through the artificial respiring part 210.

The MICOM 90 compares the tidal volume supplied, which is detected by the tidal volume detecting part 220, with the tidal volume to be supplied, and then transmits a control signal to the artificial respiring part 210 to stop the respiration supply such that the tidal volume supplied to a victim is maintained, when the compared value exceeds a predetermined range (±5%).

The respiration pressure detecting part 230 is a sensor that detects respiration pressure of the respiration oxygen supplied from the artificial respiring part 210. The respiration pressure detecting part 230 continuously detects the respiration pressure of the artificial respiring part 210 and transmits the detected value to the MICOM 90, or can transmit an alarm signal when the detected value exceeds the predetermined maximum pressure (the sum total 55 cm H20).

The respiration supply stopping part 240 immediately discharges the respiration oxygen supplied through the artificial respiring part 210 when the respiration pressure detected by the respiration pressure detecting part 230 exceeds the predetermined maximum pressure (the sum total 55 cm H20). The respiration supply stopping part 240 receives a control signal from the MICOM 90 and discharges the respiration oxygen, or can immediately discharge the respiration oxygen in response to the alarm signal of the respiration pressure detecting part 230 without the control signal of the MICOM 90, when the respiration pressure detected by the respiration pressure detecting part 230 is at the maximum pressure.

Therefore, the cardiopulmonary resuscitation apparatus of the invention prevents excessive respiration pressure from being applied to the lung by continuously checking the respiration pressure supplied to a victim.

As described above, according to the cardiopulmonary resuscitation apparatus of the invention, a sensor that detects the driving condition is provided for each component and feedback information is provided in real time to the MICOM in driving them, such that accurate and effective operations can be achieved. Further, the invention can considerably improve the performance in terms of efficiency of chest compression and artificial respiration as compared with the related art.

Although more detailed mechanical components and additional components were described in the description explaining the configuration of the invention, it should be understood that, in addition to the components of the invention describe above, peripheral mechanical components and additional components should be equipped to perform cardiopulmonary resuscitation to a victim. This is easily understood by anyone in the related field. For example, the mechanical components and additional components disclosed in Patent Registration Nos. 10-0517298, 10-0633799, or 10-0706701.

The operation of the cardiopulmonary resuscitation apparatus according to an embodiment of the invention is described hereafter. The same reference numerals as in the figures described above indicate components having the same functions. The cardiopulmonary resuscitation apparatus according to an embodiment of the invention is described, assuming that it is disposed in equipment for victim transportation, such as the board disclosed in Patent Registration No. 10-0517298. Further, it is assumed that peripheral components and additional components that are not described in the following description are corresponding components disclosed in Patent Registration No. 10-0633799 or No. 10-0706701, or other components well known in the art.

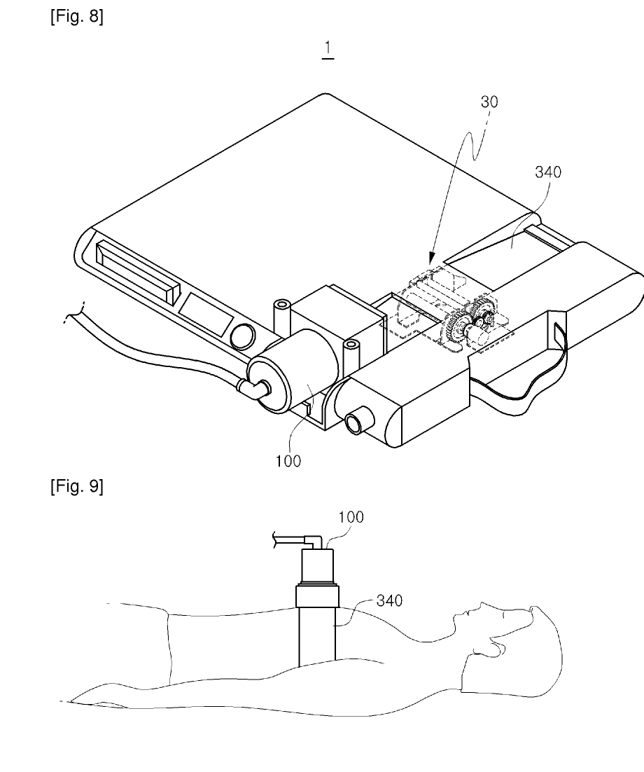

FIG. 8 is a view showing the structure of the cardiopulmonary resuscitation apparatus according to an embodiment of the invention and FIG. 9 is a view showing an example of the cardiopulmonary resuscitation apparatus according to an embodiment of the invention.

As shown in FIGS. 8 and 9, a victim is laid on the board equipped with the cardiopulmonary resuscitation apparatus 1 of the invention and the compressing unit 100 is positioned on the thorax by winding the thorax band 340 around the victim's chest.

When a button (i.e. tightening button) of the key input unit 50 is pressed to tighten the thorax band 340, a corresponding signal is transmitted to the MICOM 90 and the MICOM 90 transmits a corresponding control signal to the length adjusting unit 300 to appropriately tighten the thorax band 340. That is, the driving force is applied from the driving unit (not shown) to the pair of bobbins 310a, 320a, the length adjusting unit 300 is operated at a predetermined length by both gears 320a, 320b engaged and rotated with the pair of bobbins 310a, 310b, and the left and right thorax bands 340a, 340b are drawn in at appropriated lengths.

Thereafter, as a user selects an appropriate mode for performing cardiopulmonary resuscitation by operating the button, inputs characteristic information about the victim, and then presses a start button to start cardiopulmonary resuscitation, the MICOM 90 transmits a corresponding signal to the girth measuring unit 40 and transmits and stores the victim's girth on the basis of length difference generated when the length adjusting unit 300 adjusts the length of the thorax band 340. That is, as the length adjusting unit 300 drives the pair of bobbins 310a, 310b by actuating the driving unit (not shown), the normal/reverse rotation of the gears 320a, 320b engaged and rotated with the bobbins is transmitted to the length change measuring part 42 through the variable gear assembly 41. The length change measuring part 42 detects the normal/reverse rotation, measures and stores the length change of the thorax band 340.

The body type analyzing unit 60 classifies the body type characteristics of the victim on the basis of the characteristic information received through the key input unit 50 and reads out a corresponding inverse formula stored in the DB 80. The body type analyzing unit 60 converts the victim's girth into chest thickness and weight using the inverse formula and then stores them into the DB 80.

That is, the chest thickness converting part 61 of the body type analyzing unit 60 reads out an inverse formula for calculating the chest thickness classified and pre-calculated in the inverse formula storing part 81 of the DB 80 according to the victim's body type characteristics and the chest thickness converting part 61 calculates the victim's girth from the chest thickness using the corresponding inverse formula and stores it into the DB 80. The weight converting part 62 calculates the weight from the victim's girth and stores it into the DB 80 in the same way.

The control calculating unit 70 calculates compression depth and tidal volume on the basis of the chest thickness and weight of the victim converted by the body type analyzing unit 60, using the inverse formula stored in the DB 80, and then stores them into the DB 80.

That is, the compression depth converting part 71 of the control calculating unit 70 reads out the conversion formula for calculating pre-calculated compression depth in the conversion formula storing part 82 of the DB 80, calculates the compression depth from the victim's chest thickness obtained by the chest thickness converting part 61 using the corresponding inverse formula, and then stores it into the control information storing part 83 of the DB 80. The tidal volume calculating part 72 also calculates the tidal volume from the victim's weight obtained by the weight converting part 62 and stores it into the DB 80 in the same way.

Next, the MICOM 90 displays a variety of information of the victim stored in the DB 80 through the display unit 59 and transmits appropriate control signals such that the compressing unit 100 and the respiring unit 200 automatically perform cardiopulmonary resuscitation while maintaining appropriate chest compression and tidal volume. That is, the MICOM 90 reads out the chest compression depth stored in the control information storing part 83 of the DB 80 and correspondingly controls the compressing unit 100 to adjust the chest compression depth for the victim, while reading out the tidal volume stored in the control information storing part 83 of the DB 80 and correspondingly controlling the respiring unit 200 to maintain the amount of artificial respiration supplied to the victim.

Finally, as the user inputs an operation stop order by operating the button of the key input unit 50, the MICOM 90 stops all operations.

Further, according to the cardiopulmonary resuscitation apparatus 1 of the invention, when the user selects the cardiopulmonary resuscitation mode by operating the button in the key input unit 50, a corresponding signal is inputted to the MICOM 90, and the MICOM 90 can control cardiopulmonary resuscitation that performs chest compression and artificial respiration, which are the basic cardiopulmonary resuscitation, at a predetermined ratio (e.g. chest compression (thirty times): artificial respiration (two times)).

Further, according to the cardiopulmonary resuscitation apparatus 1 of the invention, when the user selects the continuous chest compression mode by operating the button on the key input unit 50, a corresponding signal is inputted into the MICOM 90, and the MICOM 90 can control continuous chest compression that continuously compresses only the victim's chest.

Further, according to the cardiopulmonary resuscitation apparatus 1 of the invention, when the user selects the respiration mode by operating the button on the key input unit 50, a corresponding signal is inputted into the MICOM 90, and the MICOM 90 controls respiration that performs only the artificial respiration in the operation of continuous chest compression and artificial respiration for the victim at a predetermined ratio while stopping the chest compression.

Therefore, according to the invention, it is possible to analyzing certain body type information of a victim using only some body information, and perform quickly and accurately cardiopulmonary resuscitation because optimized chest compression depth and tidal volume can be automatically adjusted according to the body type information of the victim.

A method of cardiopulmonary resuscitation according to an embodiment of the invention is described hereafter with reference to the accompanying drawings.

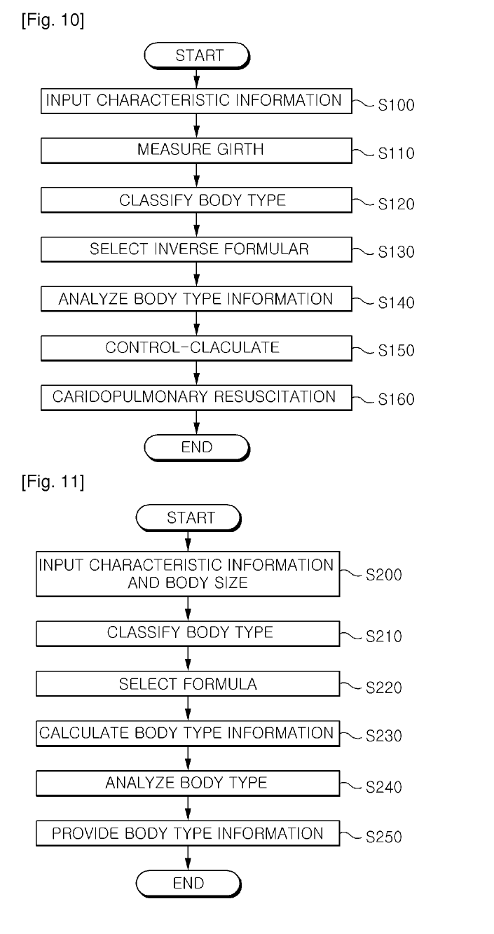

FIG. 10 is a flowchart illustrating a method of cardiopulmonary resuscitation according to an embodiment of the invention.

As shown in the figure, the cardiopulmonary resuscitation apparatus 1 first receives characteristic information about a victim through the key input unit 50 and transmits corresponding information to the MICOM 90 (S100). For example, a user inputs characteristic information that a victim is a male and teenager.

Next, the MICOM 90 of the cardiopulmonary resuscitation apparatus 1 transmits a control signal to the length adjusting unit 300 to adjust the length of the thorax band 340 and the girth measuring unit 40 measures the victim's girth from length change of the thorax band 340 and transmits the measured value to the MICOM 90 to store it into the DB 80 (S110).

Next, the body type analyzing unit 60 classifies the victim's body type on the basis of the characteristic information (S120) and then selects a pre-calculated inverse formula on the basis of the classified body type (S130). For example, when the victim is a male and teenager, the body type analyzing unit 60, as shown in Table 2, classifies the body shape into the body type A in the classification of body type and selects a corresponding inverse formula.

Thereafter, the body type analyzing unit 60 estimates each certain body type information of the victim by substituting the calculated girth to the selected inverse formula and stores the body type information into the DB 80 through the MICOM 90 (S140). That is, the chest thickness converting part 61 of the body type analyzing unit 60 calculates the chest thickness by substituting the girth value of the victim to the inverse formula corresponding to the body type A. The weight converting part 62 of the body type analyzing unit 60 calculates the weight by substituting the girth to the inverse formula corresponding to the body type A. For example, the body type analyzing unit 60 calculates the chest thickness and weight by substituting the girth value to the inverse formulae corresponding to the body type A.

Next, the control calculating unit 70 calculates appropriate compression depth and artificial tidal volume for the victim by substituting the estimated body information (chest thickness, weight) to the pre-calculated inverse formula and then stores them into the DB 80 (S150). That is, the compression depth converting part 71 of the control calculating unit 70 calculates the compression depth by substituting the chest thickness calculated by the chest thickness converting part 61 to the inverse formula. The tidal volume calculating part 72 of the control calculating unit 70 calculates the tidal volume by substituting the weight calculated by the weight converting part 62 to the inverse formula. For example, in the control calculating unit 70, when a male teenager victim of body type A has chest thickness of 186 mm and weight of 57 kg, the compressing depth converting part 71 calculates a compression depth of 46.5 mm and the tidal volume calculating part 72 calculates a tidal volume of 318.5 mHg.

Finally, the MICOM 90 controls the compressing unit 100 and the respiring unit 200 to perform cardiopulmonary resuscitation to the victim, on the basis of control information (compression depth, tidal volume) stored in the DB 80 (S160).

As described above, according to the method of cardiopulmonary resuscitation of the invention, it is possible to perform accurate and quick cardiopulmonary resuscitation because it is possible to automatically calculate body information of an unconscious victim and provide corresponding chest compression depth and tidal volume.

An analysis method of body shape according to an embodiment of the invention is described hereafter with reference to the accompanying drawings.

FIG. 11 is a flowchart illustrating an analysis method of body shape according to a preferred embodiment of the invention.

First, a person who is the object to estimate the body shape information is selected and characteristic information and some body sizes of the person is received by a direct/indirect receiving way or communication means (S200).

The characteristic information of the person may be sex and age, which is information for generally differentiating the body type. For example, the characteristic information may be male/female, teenage˜seventies, flat/middle/thick.

Further, the body sizes of the person may be one or more known body sizes, such as girth, chest thickness, weight, and height.

Next, the body type of the person is classified on the basis of the characteristic information of the person (S210) and one of the predetermined pre-calculated inverse formulae from statistics including classified body types of general people (S220).

Next, the inputted body sizes of the person is substituted in the selected inverse formulae and then certain body information of the person is calculated (S230),

where the inverse formulae are

chest thickness=k+l*girth, and

weight=m+n*girth,

(where k, l, m, and n are constants calculated according to sex and age, the chest thickness is in mm, the weight is in kg, and the other body information is in SI unit).

The inverse formulae can be obtained from functional relationship between the girth and each index, in which the indexes are C-9 (chest thickness), F-10 (girth), A-6 (height), and H-4 (weight) in ‘National Anthropometric Survey of Korea’ published by Korean Agency for Technology and Standards.

The certain body type information may be the girth, chest thickness, weight, and height.

The body type of the person is analyzed on the basis of the body information of the person calculated in step of S230 (S240).

Finally, the entire estimated body type information of the person is recorded in a database and the body type information is selectively provided if required outside (S250).

### INDUSTRIAL APPLICABILITY

As described above, according to the analysis method of body type of the invention, it is possible to calculate the entire body type information of the person by estimating other body sizes of the person from only some body information of the person.

Further, the analysis method of body type of the invention can be applied when the entire body type information (body sizes) are required from some body sizes, for example, clothes, health, and three-dimensional model etc.

Further, it is possible to provide body type information (e.g. chest thickness, girth, weight, and flatness) needed for the cardiopulmonary resuscitation according to an embodiment of the invention.

The invention is not limited to the above embodiments and can be variously modified without departing from the scope of the invention, and it should be understood that the modified scope is included in the following claims.

1. An analysis method of body type, comprising:
receiving information of characteristic information and body sizes of a person whose body type information is analyzed;
classifying the body type of the person according to the characteristic information;
selecting an inverse formula corresponding to the classified body type;
calculating body type information of the person by substituting the received body sized to the selected inverse formula; and
analyzing the body type of the person on the basis of the calculated body type information.

2. The analysis method of body type according to claim 1, wherein the characteristic information includes any one or more of sex and age.

3. The analysis method of body type according to claim 1, wherein the inverse formula is,
chest thickness=k+l*girth, and
weight=m+n*girth,
(where k, l, m, and n are constants calculated according to sex and age, unit is in SI unit).

4. The analysis method of body type according to claim 1, further comprising:
selectively providing body type information, if required outside, by storing the body type information of the person in a database.

5. A cardiopulmonary resuscitation apparatus that includes a thorax band that tightens and contracts the victim's thorax, and a length adjusting unit that adjusts length of the thorax band using normal/reverse rotation of both side bobbins that are engaged with gears connected with the thorax band, the cardiopulmonary resuscitation apparatus comprising,
a girth measuring unit that measures the victim's girth on the basis of length change of the thorax band by the length adjusting unit,
wherein the girth measuring unit includes:
a variable gear assembly that is engaged with a gear at a side of the length adjusting unit;
a length change measuring part that measures change in length of the thorax band by detecting normal/reverse rotation of the variable gear assembly; and
a girth calculating part that calculates the victim's girth on the basis of the length change of the thorax band measured by the length change measuring part.

6. A cardiopulmonary resuscitation apparatus that includes a thorax band that tightens and contracts the victim's thorax when a chest compressing unit compresses the chest, a length adjusting unit that adjusts length of the thorax band using normal/reverse rotation of both side bobbins that are engaged with gears connected with the thorax band, and a respiring unit for artificial respiration, the cardiopulmonary resuscitation apparatus comprising:
a girth measuring unit that measures the victim's girth on the basis of length change of the thorax band by the length adjusting unit;
a body type analyzing unit that selects an inverse formula corresponding the victim's characteristic information provided from the outside and analyzing the victim's body type by substituting girth to the inverse formula;
a control calculating unit that calculates appropriate compression depth and artificial tidal volume of the victim by substituting the body type information obtained by the body type analyzing unit to a corresponding conversion formula; and
a MICOM that controls the compressing unit and the respiring unit on the basis of the compression depth and tidal volume.

7. The cardiopulmonary resuscitation apparatus according to claim 6, wherein the body type analyzing unit further includes a chest thickness calculating part that calculates chest thickness by substituting the victim's chest thickness to a corresponding inverse formula.

8. The cardiopulmonary resuscitation apparatus according to claim 6, wherein the body type analyzing unit further includes a weight calculating part that calculates weight by substituting the victim's girth to a corresponding inverse formula.

9. The cardiopulmonary resuscitation apparatus according to claim 6, wherein the control calculating unit further includes a compression depth calculating part that calculates appropriate compression depth by substituting the victim's chest thickness to a corresponding conversion formula.

10. The cardiopulmonary resuscitation apparatus according to claim 6, wherein the control calculating unit further includes a tidal volume calculating part that calculates appropriate tidal volume by substituting the victim's weight to a corresponding inverse formula.

11. The cardiopulmonary resuscitation apparatus according to claim 6, wherein the inverse formula is,
chest thickness=k+l*girth, and
weight=m+n*girth,
(where k, l, m, and n are constants calculated according to sex and age, unit is in SI unit).

12. The cardiopulmonary resuscitation apparatus according to claim 8, wherein the conversion formula is,
compression depth=chest thickness*0.25, and
tidal volume=weight*6.5,
(where unit is SI unit).

13. The cardiopulmonary resuscitation apparatus according to claim 6, wherein the girth measuring unit calculates the girth using the following formula,
Y=aX2+bX+c
(where a, b, and c are measurement constants, X is DC resistance value, Y is girth, and the range is 67<=Y<=157 and 1500<=X<=4300, and unit is SI unit).

14. The cardiopulmonary resuscitation apparatus according to claim 6, wherein the girth measuring unit includes:
a variable gear assembly that is engaged with a gear at a side of the length adjusting unit,
a length change measuring part that measures change in length of the thorax band by detecting normal/reverse rotation of the variable gear assembly, and
a girth calculating part that calculates the victim's girth on the basis of the length change of the thorax band measured by the length change measuring part.

15. The cardiopulmonary resuscitation apparatus according to claim 14, wherein the length change measuring part is a potentiometer.

16. The cardiopulmonary resuscitation apparatus according to claim 6, wherein the compressing unit further includes:
a compressing part that compresses the victim's thorax;
a compression driving part that drives the compressing part; and
a compression depth detecting part that is disposed in the compression driving part and measures compression depth for compressing the victim's thorax, and the compression depth detecting part is an indirect measuring sensor.

17. The cardiopulmonary resuscitation apparatus according to claim 6, wherein the respiring unit further includes:
an artificial respiring part that discharges respiration oxygen; and
a tidal volume detecting part that is disposed in the artificial respiring part and measures the amount of discharged respiration oxygen.

18. The cardiopulmonary resuscitation apparatus according to claim 17, wherein the respiring unit further includes:
a respiration pressure detecting part that measures the pressure of the respiration oxygen discharged through the artificial respiring part; and
a respiration supply stopping part that stops respiration oxygen supply before the pressure of the respiration oxygen measured by the respiration pressure detecting part exceeds a predetermined limit.

19. A method of cardiopulmonary resuscitation using the cardiopulmonary resuscitation apparatus according to claim 6, comprising:
measuring the victim's girth on the basis of length change of the thorax band by the length adjusting unit;
receiving characteristic information about the victim;
analyzing body type of the victim by selecting a corresponding inverse formula on the basis of the characteristic information and analyzing the body type of the victim by substituting the girth to the inverse formula;
control-calculating appropriate compression depth and artificial tidal volume for the victim by substituting the body type information obtained by the body type analyzing unit to a corresponding conversion formula; and
performing cardiopulmonary resuscitation to the victim by controlling the compressing unit and the respiring unit on the basis of the compression depth and tidal volume.

20. The method of cardiopulmonary resuscitation according to claim 19, wherein the analyzing of body type further includes:
classifying the victim's body type according to the characteristic information; and
selecting a corresponding inverse formula on the basis of the classified body type.
